(19) United States
(12) Patent Application Publication
(10) Pub. No.: US 2025/0298023 A1
(43) Pub. Date: Sep. 25, 2025

(54) DIAGNOSTIC METHOD

(71) Applicant: UNIVERSITA' DEGLI STUDI DI MILANO, Milano (IT)

(72) Inventors: Tiziana BORSELLO, Milano (IT); Clara Alice MUSI, Milano (IT); Carlo BONADONNA, Milano (IT)

(73) Assignee: UNIVERSITA' DEGLI STUDI DI MILANO, Milano (IT)

(21) Appl. No.: 18/862,228

(22) PCT Filed: May 3, 2023
(86) PCT No.: PCT/IB2023/054593

§ 371 Date: Nov. 1, 2024

(30) Foreign Application Priority Data
May 4, 2022 (IT) 102022000009026

(51) Int. Cl. G01N 33/573; G01N 33/68

(52) U.S. Cl. CPC G01N 33/573; G01N 33/6896; G01N 2333/912; G01N 2440/14; G01N 2800/2814

(57) ABSTRACT

A method for diagnosing neurodegenerative diseases, the method including measuring the JNK3 levels in a biological sample selected from plasma, CSF, and saliva. The method also includes measuring P-JNK3.

The present invention relates to a non-invasive method for the early diagnosis of neurodegenerative phenomena.

### BACKGROUND ART

Synapse dysfunction is the first toxic event common to neurodegenerative and psychiatric diseases. The impairment of synaptic contacts has a devastating effect on neuronal communication, leading to wide-ranging effects such as the disruption of the neuronal networks. Moreover, several evidences from the literature report how different neurological and psychiatric disorders, ranging from mental retardation depression but also Alzheimer's disease and schizophrenia, have in common alterations in the morphology and number of dendritic spines, clear signs of synaptic dysfunction (Lepeta K et al. J Neurochem. 2016; 138:785-805). The most studied and characterized synaptopathy is that characterizing the early stages of Alzheimer's disease, which is the most widespread neurodegenerative disease but still lacks treatment today.

The early stages of AD are of particular interest, i.e., those stages of the disease which are almost asymptomatic in which the neurodegeneration process known as synaptopathy is still reversible. In particular, mild cognitive impairment (MCI) represents a transitional state between normal aging and dementia, although not all patients with MCI automatically convert to AD. Differentiating those patients with MCI due to AD is of fundamental importance so as to identify the disease early in order to intervene already in these early stages. Another confounding factor in the diagnosis of AD is frontotemporal dementia (FTD). AD and FTD are the two main forms of dementia and the differential diagnosis is often complex.

Currently, the diagnosis of Alzheimer's disease requires the presence of both the typical clinical phenotype and the classic biomarkers of the disease. Therefore, clinical assessment by cognitive tests (including Mini-Mental State Examination and Montreal Cognitive Assessment), neuroimaging, and biomarker measurements (low Aβ42 level in cerebrospinal fluid, increased Aβ40/Aβ42 ratio in cerebrospinal fluid, or high tracer retention in amyloid PET, high phosphorylated tau in cerebrospinal fluid, or increased tau PET ligand retention) are standard procedures for the complete diagnosis of AD.

These tests are capable of defining the diagnosis of declared dementia or MCI with a certain degree of certainty but have a low predictive and prognostic level on the evolution of the disease. Moreover, excluding the cognitive tests, they are all very invasive diagnostic techniques: in addition to being painful, the withdrawal of CSF does not allow close sampling over time to monitor the course of the disease; MRI and PET instead involve the injection of radioactive contrast fluids and appear to have a high cost.

Recently, in order to find new biomarkers for AD, the scientific community has described and published the Strategic Biomarker Roadmap (Boccardi et al., 2021) to validate and compare the search for new biomarkers, thus seeking to standardize methodological protocols. The guidelines divide the criteria into 5 stages/steps, including a comparison of the new biomarkers with the classic ones (Al31-40/Al31-42, Tau and PET). Compliance with these guidelines is essential to efficiently implement AD biomarkers in clinical research and diagnostics.

Certain scientific works focus on new biomarkers which can be correlated to synaptic function and dysfunction processes. Among these, SNAP-25, a pre-synaptic protein with a key role in the release of neurotransmitter vesicles (Brinkmalm et al., 2014), neurogranin (Kvartsberg et al., 2015), a post-synaptic protein involved in long-term enhancement and memory consolidation (Brinkmalm et al., 2014; Kvartsberg et al., 2015) and synaptotagmin-1, another presynaptic protein involved in neurotransmitter release (Ohrfelt et al., 2016), have been dosed at the CSF (Cerebral Spinal Fluid) level. The levels of these proteins are altered in AD and MCI with respect to controls.

An increase in the levels of mRNA as well as JNK3 protein, a protein kinase selectively expressed in the central nervous system, has been observed in brain tissues of AD patients (Wang et al., 2020). The method described is not translatable into clinical practice, where the biomarker must be measured in brain tissues.

The need for reliable early biomarkers of the onset of neurological, neurodegenerative diseases, which can be measured in a non-invasive and economical manner, remains strongly felt.

### DESCRIPTION

The present invention firstly relates to a method for the early diagnosis of neurodegenerative phenomena comprising measuring JNK3 levels in biological tissues selected from the group consisting of CSF (cerebrospinal fluid), plasma or saliva.

In an embodiment, said neurodegeneration is selected from the group comprising AD, MCI or FTD and said measurement is carried out in the CSF.

The present invention further relates to a method for the early diagnosis of neurodegenerative phenomena, which also comprises measuring active JNK3 (P-JNK3) levels. The P-JNK3/JNK3 ratio allows having an internal benchmark which normalizes for each sample the active form of the enzyme on the total enzyme present in the sample. The internal benchmark helps to minimize systematic errors between samples from different hospitals, which follow different protocols for collecting and storing patients' biological fluids.

### DESCRIPTION OF THE DRAWINGS

FIG. 1: (A) CSF JNK3 levels measured in MCI patients (n=9), AD (n=35), FTD (n=5) and Ctr control subjects (n=25). The data are shown as mean±SEM and analyzed with multiple t-tests. ***p<0.001 vs Ctr. (B) CSF JNK3 levels measured in MCI (n=18), AD (n=47), FTD (n=5) patients shown in relation to the mean recorded in the control group obtained from the same disease sample collection center and Ctr control subjects (n=38). The data are shown as mean±SEM and analyzed with multiple t-tests. *p<0.05, **p<0.01, ***p<0.001 vs Ctr.

FIG. 2: A) CSF JNK3 levels measured in MCI, AD, FTD and hydrocephalus patients (n=3). B) JNK3 was immunoprecipitated from CSF with a specific JNK3 antibody, separated on SDS-PAGE and analyzed with Western Blot with antiP-pan-JNK. Representative images are shown on samples from control subjects (left panel) or AD subjects (right panel). Immunoprecipitation with IgG antibodies was used as a control. C) Quantification of P-JNK/JNK3 ratio in the immunoprecipitated samples. *p<0.05 vs Ctr

FIG. 3: Plasma JNK3 levels measured in AD patients and control patients. The data are shown as mean±SEM and analyzed with t-tests. *p<0.05 vs Ctr.

FIG. 4: Dosage of JNK3 in the saliva of three healthy patients.

The authors of the present invention collected biological samples from AD subjects, FTD subjects, MCI subjects and hydrocephalus subjects, as well as from control subjects.

In the samples thus obtained, the JNK3 levels were measured with methods and antibodies known to those skilled in the art.

The data obtained are capable of classifying the subjects, where the JNK3 levels are significantly increased in subjects suffering from nervous system diseases with respect to control subjects. Said diseases underlie a synaptic dysfunction.

Surprisingly, the increase in JNK3 was observed, and to a significantly greater extent, also in FTD, MCI and hydrocephalus subjects.

Therefore, the present invention firstly relates to a method for diagnosing and monitoring the treatment of nervous system diseases, where said method comprises:
providing a biological sample, selected from the group consisting of CSF, plasma, saliva from a subject under analysis, and optionally from at least one control subject, where said control subject is a subject not suffering from any nervous system disease, preferably homogeneous by sex and age with said subject under analysis;
measuring the JNK3 levels in said biological samples;
classifying said subject as suffering from a nervous system disease, when said JNK3 level measured in said sample from said subject under analysis is significantly greater than the level measured in control subjects.

In an embodiment, said nervous system diseases are selected from the group comprising AD, FTD, MCI and hydrocephalus.

In an embodiment, when measured in the CSF, the control JNK3 levels are equal to or less than 1,900 ng/L. JNK3 levels between 1,900 ng/l and 2,400 ng/l measured in the CSF classify said subject as an AD subject; JNK3 levels above 2,500 ng/l measured in the CSF classify said subject as a subject suffering from FTD, MCI, or hydrocephalus.

In an embodiment, when measured in plasma, the control JNK3 levels are equal to or less than 160 pg/ml. JNK3 levels measured in plasma above 160 pg/mL classify said subject as a subject suffering from AD.

In an embodiment, which excludes the variability due to the experimental setting, said JNK3 values are shown in relation to the value measured from a control sample, collected and analyzed in parallel. In this embodiment, increases equal to or greater than 1.4 times the value measured in the control are indicative of a nervous system disease.

Said method, when used for monitoring the progression of a disease/therapeutic treatment, has the JNK3 level as a control, measured in the same biological sample obtained from the same subject at a time prior to the time in which the analysis is being carried out. By way of non-limiting example, where the first measurement is carried out on a 50-year-old subject, said JNK3 levels measured in said biological sample from said subject will be defined as t1 levels. At later times, for example one year later, the measurement is repeated, obtaining the values at t2, t3 . . . tn. A curve is thus constructed, which collects said values from t1 to tn, where an ascending curve is indicative of a phenomenon of synaptic degeneration. After reaching the peak, the curve becomes decreasing to indicate the process of neuronal death and irreversible progression of the disease, or lack of efficacy of the current treatment. Conversely, a curve which tends not to increase is indicative of a situation of substantial stability or inhibition of the synaptic dysfunction process, therefore of efficacy of the therapeutic treatment.

In a further aspect, a method is claimed herein comprising, in addition to the measurement of JNK3, also the measurement of P-JNK3 levels in the same biological sample. Phosphorylated JNK3 (P-JNK3) is the active form of JNK3 kinase and the presence thereof is indicative per se of a tissue stress phenomenon.

In this embodiment, the value considered for the purposes of the above classification is P-JNK3/JNK3. Levels of said P-JNK3/JNK3 ratio above the levels of the P-JNK3/JNK3 control ratio are indicative of nervous system disease.

In an embodiment, said P-JNK3 measurement is carried out with immunoprecipitation assays, for example by immunoprecipitating with an anti-JNK3 antibody and highlighting the band after running on SDS-PAGE with anti-P-JNK antibody. In a further embodiment, said measurement is carried out with a double ELISA assay.

The dosage of the active form of JNK3 on the total form i.e., the P-JNK3/JNK3 ratio allows, within each experiment or set of experiments, to have an internal standard which avoids errors in measurement and normalization, thus providing an internal control of the methodology, increasing the diagnostic reliability thereof. This is a fundamental point since the problem of variability and the lack of a common and universal biological sample management protocol between the different hospital centers, the variability of the results obtained by the different operators and the heterogeneity of the different biological fluids examined are issues considered primary in clinical research, as highlighted in the Strategic Biomarker Roadmap (Boccardi et al., 2021).

The following examples have the mere purpose of illustrating the invention, without limiting in any manner. The scope of the invention is defined by the following claims.

### EXAMPLES

### Example 1: JNK3 Measurement in MCI, AD and FTD Patients

CSF samples were collected from patients with AD, frontotemporal lobar dementia (FTD), and mild cognitive impairment (MCI), and from non-dementia controls. In the samples, JNK3 levels were measured with a commercially available ELISA kit (Human Stress-activated protein kinase JNK3, MAPK10 ELISA kit #BTB-E6701HU).

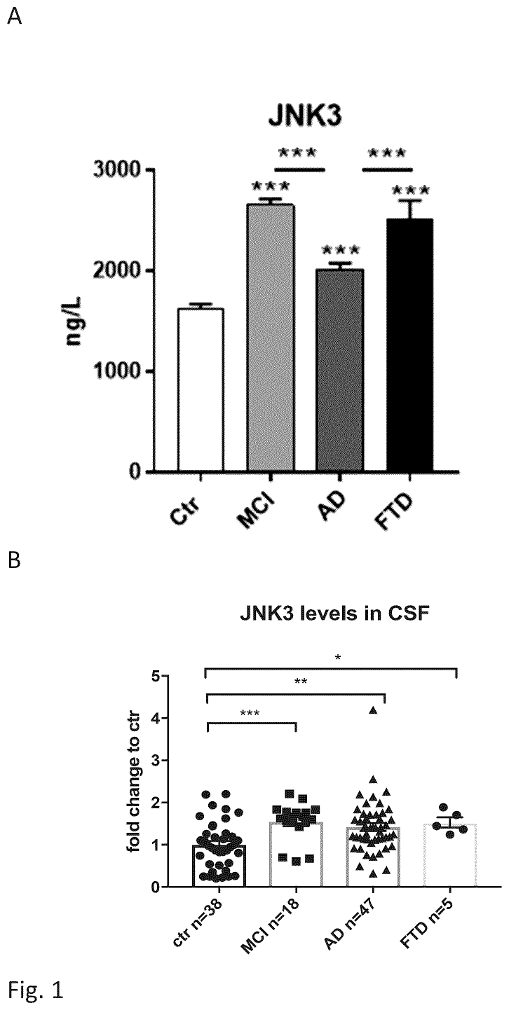

The following samples were tested in this first cohort: non-dementia controls Ctr (n=25), MCI patients (n=9), AD patients (n=35), FTD patients (n=5). The results are shown in the graph in FIG. 1A.

The control group showed an average JNK3 level of 1620 ng/L; the MCI group showed a significant increase in JNK3 with respect to ctr, with an average JNK3 level of 2660 ng/L (164% of the control group; p<0.0001); the Alzheimer group also showed a significant increase in JNK3, with 2020 ng/L (125% of the control group; p<0.0001); the group with frontotemporal dementia showed an increase in JNK3, compared to ctr, with an average of 2510 ng/L (154% of the control; p<0.0001).

The experiment was repeated for appropriate validation on a different cohort of subjects. CSF samples were collected from patients with AD, FTD and MCI, and from non-dementia controls from 3 different hospital facilities. The JNK3 levels were measured in the samples with the ELISA kit already used in example 1.

The following samples were tested in this second cohort: non-dementia controls Ctr (n=38), MCI patients (n=18), AD patients (n=47), FTD patients (n=5).

Since the samples come from different hospitals, the analysis of JNK3 levels was carried out by relating the values measured in the different patient groups (MCI, AD, FTD) to the average recorded in the control group for each collection center, thus evaluating the variation of JNK3 levels in relation to the controls. The results are shown in the graph in FIG. 1B as a fold-change with respect to the control group±SEM.

The control group is normalized at 1±0.09121 (SEM). The MCI group showed a significant increase in JNK3 with respect to ctr of 1.548 times±0.1064 (p=0.0007); the Alzheimer group also showed a significant increase in JNK3 of 1.421 times±0.09286 (p=0.0020); the group with frontotemporal dementia showed an increase in JNK3, with respect to ctr, of 1.529 times±0.2571 (p=0.046).

### Example 2: P-JNK3 measurement in Cerebrospinal Fluid

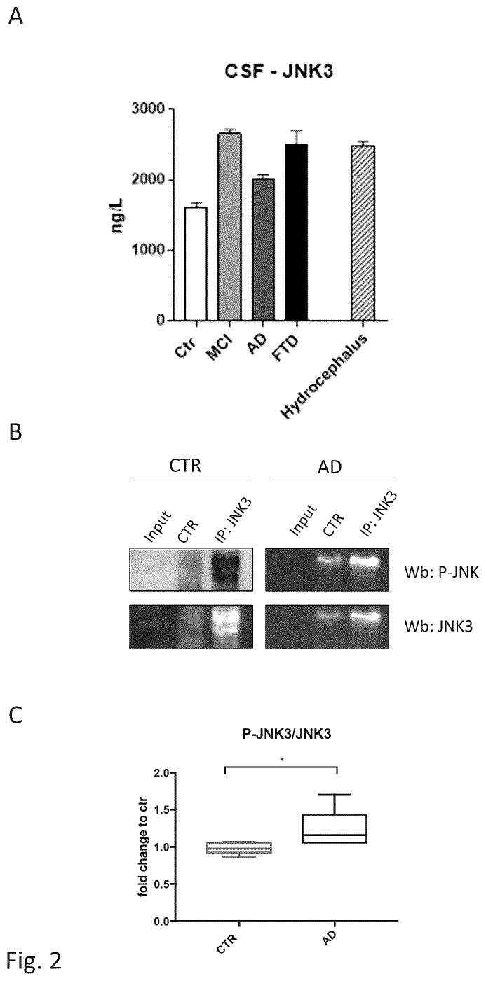

Since JNK3 is the most reactive JNK isoform, it is very important to determine if it is in an active state in CFS, i.e., in the form of P-JNK3. For this purpose, immunoprecipitation analyses were performed on CSF samples obtained from AD patients and related controls. Due to the lack of a specific antibody capable of recognizing only the phosphorylated form of JNK3 among other isoforms, JNK3 was immunoprecipitated and then labeled with P-JNK to assess the amount of phosphorylated JNK3 in the immunoprecipitate. These results indicated that a large amount of immunoprecipitated JNK3 is phosphorylated. These data show for the first time that JNK3 is phosphorylated and thus appears to be an active enzyme in CFS. The data are shown in FIG. 2B. It is apparent from the images and the quantification thereof (FIG. 2C) that the P-JNK3/JNK3 ratio is significantly higher in AD than in the age-matched controls. Said P-JNK3/JNK3 ratio allows having an internal standard, making it an ideal biomarker for early detection.

### Example 3: JNK3 Measurement in Plasma

Plasma samples were collected from AD patients and non-dementia controls. JNK3 levels were measured in the samples with a commercially available ELISA kit.

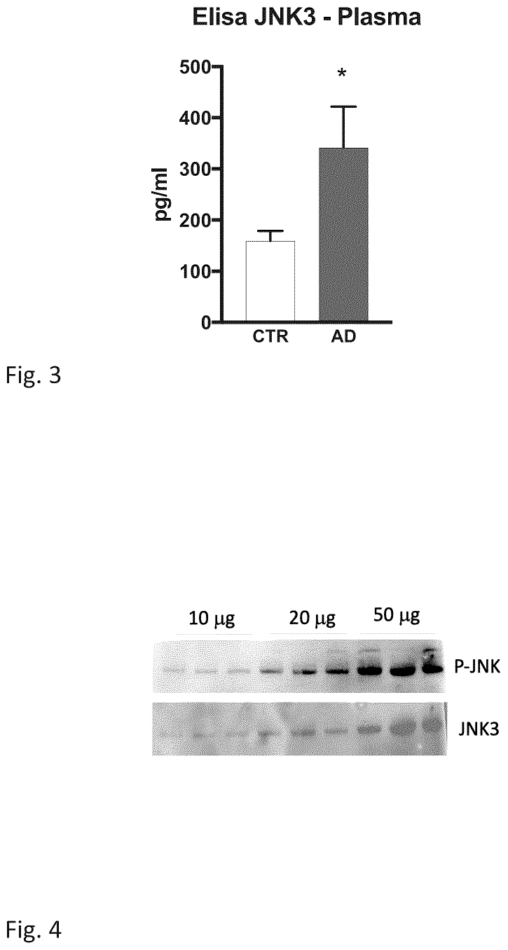

The following samples were tested: non-dementia controls Ctr (n=10) and AD patients (n=10). The results are shown in the graph in FIG. 3.

The control group showed an average JNK3 level of 159.4 pg/ml; the Alzheimer's group showed a significant increase in JNK3, with 342 pg/ml (46% of the control group; p<0.05).

### Example 4: P-JNK and JNK3 Measurement in Saliva

Saliva samples were collected from non-dementia controls. The JNK3 and P-JNK3 levels were measured in the samples with Western Blot biochemical analysis, demonstrating the possibility of dosing JNK3 and P-JNK3 in saliva as well. The data are shown in FIG. 4.

1-12. (canceled)

13. A method for diagnosing and monitoring the treatment of diseases of the nervous system and synaptic dysfunction, the method
comprising:
providing a biological sample selected from the group consisting of: plasma, CSF, saliva from a subject under analysis, and from at least one control subject, wherein said control subject is a subject not suffering from any neurodegenerative disease;
measuring the JNK3 levels in said biological samples; and
classifying said subject under analysis as suffering from a nervous system disease, when said JNK3 level measured in said sample from said subject under analysis is higher than a control level.

14. The method according to claim 13, wherein said disease of the nervous system and synaptic dysfunction is chosen from the group consisting of: Alzheimer's (AD), Frontotemporal Dementia (FTD), and mild cognitive impairment (MCI).

15. The method according to claim 13, wherein said biological sample is CSF.

16. The method according to claim 15, wherein JNK3 levels above 1,900 ng/I measured in said subject under analysis are indicative of a nervous system disease.

17. The method according to claim 14, wherein JNK3 levels between 1,900 ng/I and 2,400 ng/I measured in said subject under analysis classify said subject as an AD subject.

18. The method according to claim 14, wherein JNK3 levels above 2,500 ng/I classify said subject as a subject suffering from FTD, MCI or hydrocephalus.

19. The method according to claim 13, wherein at least a 1.4-fold increase in JNK3 values with respect to the values measured in the control is indicative of a central nervous system disease.

20. The method according to claim 13, wherein said biological sample is plasma.

21. The method according to claim 20, wherein levels above 160 pg/ml classify said subject as a subject suffering from AD.

22. The method according to claim 13, further comprising:
measuring the P-JNK3 levels in said biological sample, and
defining the P-JNK3/JNK3 ratio,
wherein a P-JNK3/JNK3 ratio measured in the sample under analysis which is greater than the P-JNK3/JNK3 ratio measured in the control
classifies the subject under analysis as suffering from a nervous system disease.

23. The method according to claim 22, wherein said nervous system disease is AD.

24. The method according to claim 13, wherein said biological samples from said subjects under analysis are taken and measured repeatedly over time.
